(19) United States
(12) Patent Application Publication
(10) Pub. No.: US 2008/0183048 A1
(43) Pub. Date: Jul. 31, 2008

(54) Electronic timed caller mattress

(76) Inventor: Lily Zhang, Brookfield, WI (US)

Correspondence Address: RYAN KROMHOLZ & MANION, S.C., POST OFFICE BOX 26618, MILWAUKEE, WI, 53226, US

(21) Appl. No.: 12/011,242

(22) Filed: Jan. 25, 2008

Related U.S. Application Data
(60) Provisional application No. 60/898,197, filed on Jan. 29, 2007.

(51) Int. Cl. A61G 7/00; A61B 5/00

(52) U.S. Cl. 600/300; 5/600

(57) ABSTRACT

A system for monitoring the position of a patient and alerting a caregiver of the need to move the patient is provided, including a sensing device, a controller and at least one alerting device. The sensing device is located on top of or within the patient's mattress and sends signals regarding the patient's position to the controller. The controller analyzes the position signals and stores the information. If the patient is not moved within a predetermined time period, the controller sends a signal to the alerting device carried by the caregiver.

### RELATED APPLICATIONS

This application claims the benefit of co-pending U.S. Provisional Patent Application Ser. No. 60/898,197, filed 29 Jan. 2007.

### BACKGROUND OF THE INVENTION

An important aspect of caring for long-term bedridden patients is turning over the patients on schedule. Bedsores, also known as pressure ulcers or decubitus, are lesions caused by unrelieved pressure to any part of the body. If bedridden patients are not turned on schedule pressure ulcers can form on the patient's body. Pressure ulcers range in severity, but may require surgical intervention to treat. Pressure ulcers not only cause unnecessary psychological and physical suffering to patients, but also increase the medical costs of treating patients.

Nursing or other attending staff in a hospital, nursing home, or other care facility may work under conditions involving high pressure, high stress, and long hours. Under these conditions, along with various shift changes, it can become difficult to keep track of which patients have been turned over.

### SUMMARY OF THE INVENTION

The present invention provides an electronic timed caller mattress system. The system includes a sensing device with a plurality of sensors, a controller in electronic communication with the sensing device, and at least one alerting device in electronic communication with the sensing device.

The controller may be adapted to analyze data received from the sensing device and send signals to the at least one alerting device based on the analysis of said data.

The system may further include a data display device in electronic communication with the controller. The system may further include a data input device in electronic communication with the controller.

The system may further include a transmitting device in electronic communication with the controller. The transmitting device may be in electronic communication with the at least one alerting device. The electronic communication between the transmitting device and the at least one alerting device may be wireless.

The system may include sensors which are pressure sensitive sensors. The system may include sensors which are elastic members.

The sensing device may be a mattress. The sensing device may be a pad adapted to be placed on top of a mattress. The alerting device may be a mobile electronic device.

One embodiment of the invention provides a method including providing a sensing device with a plurality of sensors, a controller in electronic communication with the sensing device, and at least one alerting device in electronic communication with the sensing device. The method further includes collecting data from at least one sensor. The method further includes sending the data to controller. The method further includes analyzing the data. The method further includes storing the data.

The analyzing data step may further include comparing the data to previously stored data. The method may further include determining whether the data has changed in a predetermined time alarm period.

The method may further include sending a signal to an alerting device if the data has not changed in a predetermined time alarm period.

### BRIEF DESCRIPTION OF THE DRAWINGS

FIG. 1 is a simplified diagram of the present invention.

FIG. 2 is a flow chart of the steps in the operation of an embodiment of the present invention.

FIG. 3 is a simplified perspective view of an embodiment of a sensing device according to the present invention.

FIG. 4 is a simplified representation of the third layer of the sensing device shown in FIG. 3.

FIG. 5 is a simplified representation of the fourth layer of the sensing device shown in FIG. 3.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

The present invention comprises a system and apparatus to constantly monitor the state of bedridden patients and electronically alert nurses or care givens for proper response.

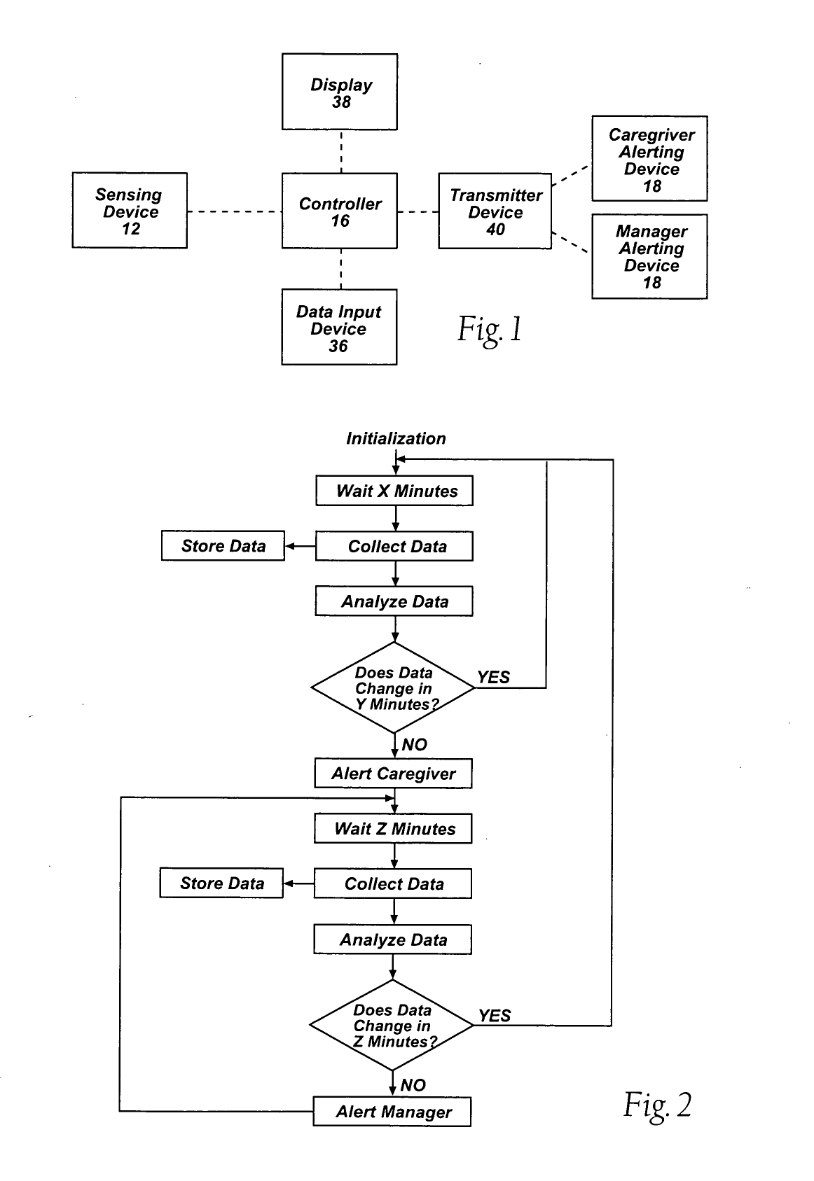

As shown in FIG. 1, the system preferably includes a sensing device 12 with a plurality of sensors 14 coupled to a controller 16. The controller 16 processes and analyzes signals collected from the sensors 14. The signals collected from the sensors 14 indicates the particular position of a patient and the period of time the patient has been in that position through timing, sampling, and comparing the data from the sensors 14. If the time exceeds a predetermined and preset parameter, the controller 16 will send a signal to an altering device 18 to alert a caregiver that care must be given to the patient. This reminder to the caregiver may prevent the outbreak of pressure ulcers and other related problems and ultimately improve the quality of patient care.

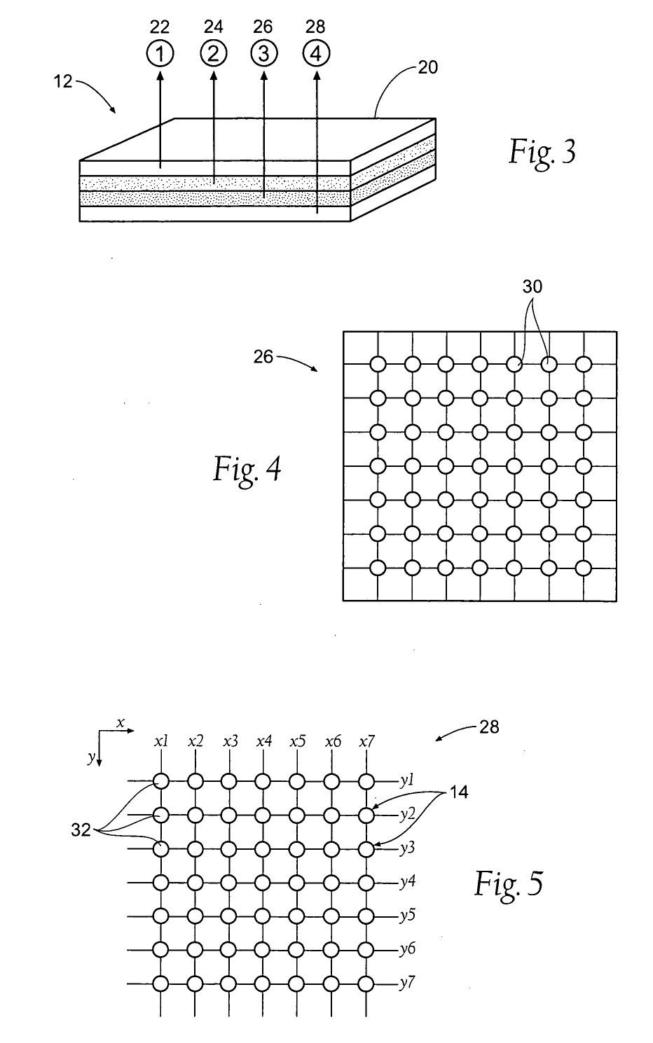

In the illustrated embodiment shown in FIG. 3, the sensing device 12 takes the form of a specialized mattress 20 which may include a mattress cover 22, a sponge mat 24, a reticulated frame 26, and a distributing testing area/position 28.

The mattress cover 22 is preferably of a pliable material that is moisture proof, wears well, and is easy to clean.

The sponge mat 24 is preferably break and pressure resistant. The sponge mat 24 is preferably pliable and ply fitting.

The reticulated frame 26 is preferably made of a metal material. The reticulated frame 26 is preferably pressure and distortion resistant. As shown in FIG. 4, the reticulated frame 26 includes a plurality of generally round shaped holes 30 which are the popout positions for testing the pressure resistance of the elastic component in order to prevent the depth distortion of elastic component when the elastic component comes under pressure. It is contemplated that the reticulated frame 26 may be divided into three sections, and connected with joints. The middle section's frame may use multiple joints to make the frame flexible to fit into a medical bed which allows the head/foot of the mattress to be elevated/put down freely with the medical bed function together.

The distribution testing area 28 may include a plurality of elastic components 32. When the elastic components 32 are pushed down, the corresponding position (Xi and Yj) will be detected and sent to the controller 16.

It is also contemplated that the sensing device 12 could comprise a pad to be placed over a standard mattress, rather than being built into the mattress 20 itself as described above.

It is further contemplated that the reticulated frame layer 26 could be eliminated and the distribution testing area 28 could comprise a plurality of spaced apart pressure sensitive sensors 14.

The sensing device 12 is preferably in electronic communication, either directly or indirectly, with an electronic control circuit 34. The electronic communication may be wired or wireless.

The electronic control circuit 34 preferably includes a controller 16, a data input device 36 36, a data display device 38, and an electrical source (not shown). The electronic control circuit 34 could be built into a single device, or may be separate components.

The controller 16 may take any form known in the art including, but not limited to a computer or microprocessor. For example, the controller 16 may utilize a device such as C8051F020 from Silicon Laboratories, Inc. The controller 16 is preferably adapted to store and analyze the data received from the sensors 14.

It is contemplated that each sensing device 12 could have its own controller 16 associated with it, or that multiple sensing devices 12 could communicate with a single controller 16. For example, in a nursing home or other patient care facility there could be a controller 16 in each patient room, on each floor, or a single controller 16 for the entire facility.

The controller 16 may be set up to record any information desirable to the particular circumstances of the facility. For example, the controller 16 may record information such as the date, time, room number, bed number, location, sensing device identifier, patient identifier, caregiver identifier, and manager identifier. The controller 16 may also be set up to be adjustable. For example, the data collection interval, the alarm interval, and the correction interval may be amended based on the needs of facility.

The system preferably includes a data input device 36 coupled to the controller 16. The data input device 36 may take the form of a keyboard. The data input device 36 may be used to enter set the parameters of the monitoring system and enter user information into the system. For example, the data collection interval, the alarm interval, and the correction interval may be set. It may also be desirable to allow for starting the system, resetting the system, and deleting data. The data input device 36 may also be used to enter information relating to the patient and the caregiver. For example, it may be desirable to utilize the data input device 36 to input the location of each sensing device 12, the patient associated with that device, and any other data relating to the patient. It may also be desirable to input information relating to the caregiver. For example, inputting information identifying the caregiver and the required information to allow the control circuit to send a signal to the caregiver's alerting device 18.

The system preferably includes a data display device 38 coupled to the controller 16. The data display device 38 may take the form of a LCD or GUI. The data display device 38 may simulate the data related to the patient's position on the sensing device 12 and record tracking of the transacting result.

The electronic control circuit 34 preferable includes at least one power source (not shown). The electronic control circuit 34 could utilize a single power source for all items needing power, or multiple power sources for the items needing power. The electronic control circuit 34 may obtain power through any means, including but not limited to at least one batter, the electrical system in the building, or a generator.

The system preferably includes a transmitting device 40 for sending information from the controller 16 to the alerting device 18 of a caregiver. In the illustrated embodiment, the transmitting device 40 is a wireless transmitter, however it is contemplated that alternate transmitting devices 40 may be utilized. The wireless transmitter may be of any type known in the industry. It is contemplated that the data could be GPRS (general packet radio services). For example, the transmitting device 40 could utilize a devices such as the Siemens MC39I module with GPRS (general packet radio services) function. It is contemplated that the alterting device 18 could be a telephone, pager, or other standard mobile device and the transmitting device 40 could be a call machine.

It is also contemplated that it may be desirable to have a transmitting device 40 adapted to send the data collected by the sensors 14 in the sensing device 12 to the controller 16. This transmitting device 40 may be built into the sensing device 12, or may be separate from the sensing device 12.

The system preferably includes an alerting device 18. The alerting device 18 is preferably adapted to notify the caregiver that the predetermined time period for turning the patient has expired. This notification may be given to the caretaker by the alerting device 18 producing a sound, a light, or vibration.

The alerting device 18 is preferably in electronic communication with the controller 16. The electronic communication may be wired or wireless and may be direct or indirect. In the illustrated embodiment a transmitter associated with the controller 16 sends signal either directly to the alerting device 18 or to a wireless receiver associated with the alerting device 18.

The alerting device 18 preferably includes a display. In this manner, specific information to provide patient treatment, including, but not limited to, room number, bed number, time alert was sent to the alerting device 18, location of the patient, etc may also be transmitted to the alerting device 18.

The system may include a second alerting device 18 to be carried by the manager of the caregiver. The second alerting device 18 may be adapted to either receive the same signal as the first alerting device 18, or to receive a signal if the patient is not moved in a predetermined amount of time after the first alerting device 18 receives its notice.

As described above, the system is controlled by a controller 16 which is preferably programmed to receive data from the patient bed sensors 14, analyze and store the sensor data, and send signals to the caregiver alerting device 18, and, if necessary, the manager alerting device 18. FIG. 2 is a flowchart which shows the sequence in which the system operates.

The program is first initiated, preferably through the data input device 36. The controller 16 then collects data regarding the position of the patient from the sensors 14 at predetermined data collection intervals (X). For example, the data collection interval could be 30 minutes. The data is then stored. The newly collected data is then compared to the previously stored data. If the data has changed in the alarm interval (Y), the system then goes back to the data collection step and the patient position data is recollected and re-compared at the appropriate data collection interval. If the data has not changed in the predetermined alarm interval, the controller 16 sends a signal to the caregiver alerting device 18.

The system then waits a predetermined correction interval (Z), and recollects patent position data, if the patient position data has changed, the system goes back to the data collection step. If the patient-position has not changed, the controller 16 sends a signal to the manager alerting device 18. The system then waits a predetermined period of time referred to as the “correction interval” and recollects patient position data. If the patient position data has changes, the system goes back to the data collection step. If the patient position has not changed, the controller 16 repeats the signal to the manager alerting device 18.

In use, the sensing device 12 is placed underneath a patient. The caregiver associated with the patient is given an alerting device 18 to carry with them during their shift. When necessary, a message would be sent to the alerting device 18 from the electronic control circuit. As stated above, this message may include information about the location and status of the patient. Upon receiving the signal, the caregiver may attend to the needs of the patient, including changing the position of the patient.

It may be desirable for the information sent to the alerting device 18 to be stored for a predetermined amount of time. In this manner, if a problem arises because a patient was not properly turned, the information stored in the alerting device 18 may be accessed to determine the cause of the problem.

It may also be desirable for the alerting device 18 to record the code name of the caregiver on duty when any signals are sent as well as the duty time.

The manager alerting device 18 allows the manager to step in and assure proper care is provided to the patient if the signal sent to the caregiver alerting device 18 is not responded to.

Preferably, the system may be utilized to monitor several patients at one time.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. A system comprising
a sensing device with a plurality of sensors;
a controller in electronic communication with the sensing device;
and at least one alerting device in electronic communication with the sensing device.

2. The system of claim 1 wherein the controller is adapted to analyze data received from the sensing device and send signals to the at least one alerting device based on the analysis of said data.

3. The system of claim 2 further comprising a data display device in electronic communication with the controller.

4. The system of claim 2 further comprising a data input device in electronic communication with the controller.

5. The system of claim 2 further comprising a transmitting device in electronic communication with the controller.

6. The system of claim 5 wherein the transmitting device is further in electronic communication with the at least one alerting device.

7. The system of claim 6 wherein the electronic communication between the transmitting device and the at least one alerting device is wireless.

8. The system of claim 2 wherein the sensors comprise pressure sensitive sensors.

9. The system of claim 2 wherein the sensors comprise elastic members.

10. The system of claim 2 wherein the sensing device comprises a mattress

11. The system of claim 2 wherein the sensing device comprises a pad adapted to be placed on top of a mattress.

12. The system of claim 2 wherein the alerting device is a mobile electronic device.

13. A method comprising
providing a sensing device with a plurality of sensors, a controller in electronic communication with the sensing device, and at least one alerting device in electronic communication with the sensing device;
collecting data from at least one sensor;
sending data to controller;
analyzing data; and
storing data.

14. The method of claim 13 where in the analyzing data step further comprises comparing the data to previously stored data.

15. The method of claim 14 further comprising
determining whether the data has changed in a predetermined alarm time period.

16. The method of claim 15 further comprising
sending a signal to an alerting device if the data has not changed in a predetermined alarm time period.

17. The method of claim 16 further comprising
waiting a predetermined correction time period;
collecting data from at least one sensor;
sending data to controller;
determining whether the data has changed in a predetermined correction time period.

18. The method of claim 17 further comprising
providing a second alerting device; and
sending a signal to the second alerting device if the data has not changed in a predetermined correction time period.

19. A method comprising the steps of
(a) providing a sensing device with a plurality of sensors, a controller in electronic communication with the sensing device, and a first caregiver alerting device in electronic communication with the controller and a second manager alerting device in electronic communication with the controller, and proceeding to (b);
(b) waiting a predetermined data collection period and proceeding to (c);
(c) collecting new data from at least one sensor, sending the new data to the controller, and saving the new data and proceeding to (d);
(d) determining whether the data has changed in a predetermined alarm period, proceeding to (b) if the data has changed in a predetermined alarm time period and proceeding to (e) if the data has not changed in a predetermined alarm time period;
(e) sending a signal from the controller to the caregiver alerting device.

20. The method of claim 19 further comprising the steps of
(f) waiting a predetermined correction time period and proceeding to (g)
(g) collecting new data from at least one sensor, sending the new data to the controller, and saving the new data and proceeding to (h);
(h) determining whether the data has changed in a predetermined correction time period, proceeding to (b) if the data has changed in a predetermined correction period and proceeding to (i) if the data has not changed in a predetermined correction time period;
(i) sending a signal from the controller to the manager alerting device, and proceeding to (f).
